(19) United States
(12) Patent Application Publication
(10) Pub. No.: US 2017/0260153 A1
(43) Pub. Date: Sep. 14, 2017

(54) Use of Reactants in the Production of 2,5-Furandicarboxylic Acid

(71) Applicant: BP Corporation North America Inc., Naperville, IL (US)

(72) Inventors: Victor A. Adamian, Naperville, IL (US); Joseph B. Binder, San Diego, CA (US); Ryan Shea, Naperville, IL (US)

(73) Assignee: BP Corporation North America Inc., Houston, TX (US)

(21) Appl. No.: 15/517,571

(22) PCT Filed: Oct. 7, 2015
(86) PCT No.: PCT/US2015/054514

§ 371 Date: Apr. 7, 2017

Related U.S. Application Data
(60) Provisional application No. 62/061,848, filed on Oct. 9, 2014.

(51) Int. Cl. C07D 307/68

(52) U.S. Cl. CPC C07D 307/68

(57) ABSTRACT

Methods for providing effective, efficient and convenient ways of producing 2,5-furandicarboxylic acid are presented. In addition, compositions of 2,5-furandicarboxylic acid including 2,5-furandicarboxylic acid and at least one byproduct are presented. In some aspects, 4-deoxy-5-dehydroglucaric acid is dehydrated to obtain the 2,5-furandicarboxylic acid. A solvent, catalyst, and/or reactant may be combined with the 4-deoxy-5-dehydroglucaric acid to produce a reaction product including the 2,5-furandicarboxylic acid. In some arrangements, the reaction product may additionally include water and/or byproducts.

### CROSS-REFERENCE

This application claims the benefit of U.S. provisional patent application Ser. No. 62/061,848 filed Oct. 9, 2014, and entitled “Use of Reactants In the Production of 2,5-Furandicarboxylic Acid,” which is hereby incorporated herein by reference in its entirety.

### BACKGROUND

2,5-furandicarboxylic acid (FDCA) and FDCA esters are recognized as potential intermediates in numerous chemical fields. For instance, FDCA is identified as a prospective precursor in the production of plastics, fuel, polymer materials, pharmaceuticals, agricultural chemicals, and enhancers of comestibles, among others. Moreover, FDCAs are highlighted by the U.S. Department of Energy as a priority chemical for developing future “green” chemistry.

### SUMMARY

The following presents a simplified summary in order to provide a basic understanding of some aspects of the disclosure. The summary is not an extensive overview of the disclosure. It is neither intended to identify key or critical elements of the disclosure nor to delineate the scope of the disclosure. The following summary present some concepts of the disclosure in a simplified form as a prelude to the description below.

Aspects of the disclosure provide effective, efficient, and convenient ways of producing 2,5-furandicarboxylic acid (FDCA). In particular, certain aspects of the disclosure provide techniques for dehydrating 4-deoxy-5-dehydroglucaric acid (DDG) to obtain FDCA. The dehydration reaction proceeds by combining one or more reactants with a DDG starting material. One or more catalysts and/or one or more solvents may also be combined with the reactants and DDG. In some instances, the reactant may act as a dehydrating agent and may interact with hydroxyl groups on the DDG thereby encouraging elimination reactions to form FDCA. The reactant may assist the dehydration reaction thereby producing increased yields of FDCA.

In a first embodiment, a method of producing FDCA includes bringing DDG into contact with a solvent in the presence of a catalyst selected from an activated carboxylic acid derivative, activated sulfonic acid derivative, carboxylic acid halide, a ketene, and a combination thereof, and allowing DDG, the solvent, and the catalyst to react with each other to produce FDCA, any byproducts, and water. An activated acid derivative, as used herein, refers to a form of an acid which is more reactive in acyl substitution reaction than the acid itself.

These features, along with many others, are discussed in greater detail below.

### DETAILED DESCRIPTION

Various examples, aspects, and embodiments of the inventive subject matter disclosed here are possible and will be apparent to the person of ordinary skill in the art, given the benefit of this disclosure. In this disclosure reference to “certain exemplary embodiments” or aspects (and similar phrases) means that those embodiments or aspects are merely non-limiting examples of the subject matter and that there likely are other alternative embodiments or aspects which are not excluded. Unless otherwise indicated or unless otherwise clear from the context in which it is described, alternative elements or features in the embodiments and examples below and in the Summary above are interchangeable with each other. That is, an element described in one example may be interchanged or substituted for one or more corresponding elements described in another example. Similarly, optional or non-essential features disclosed in connection with a particular embodiment or example should be understood to be disclosed for use in any other embodiment of the disclosed subject matter. More generally, the elements of the examples should be understood to be disclosed generally for use with other aspects and examples of the products and methods disclosed herein. A reference to a component or ingredient being operative, i.e., able to perform one or more functions, tasks and/or operations or the like, is intended to mean that it can perform the expressly recited function(s), task(s) and/or operation(s) in at least certain embodiments, and may well be operative to perform also one or more other functions, tasks and/or operations.

While this disclosure includes specific examples, including presently preferred modes or embodiments, those skilled in the art will appreciate that there are numerous variations and modifications within the spirit and scope of the invention as set forth in the appended claims. Each word and phrase used in the claims is intended to include all its dictionary meanings consistent with its usage in this disclosure and/or with its technical and industry usage in any relevant technology area. Indefinite articles, such as “a,” and “an” and the definite article “the” and other such words and phrases are used in the claims in the usual and traditional way in patents, to mean “at least one” or “one or more.” The word “comprising” is used in the claims to have its traditional, open-ended meaning, that is, to mean that the product or process defined by the claim may optionally also have additional features, elements, steps, etc. beyond those expressly recited.

### Dehydration Reaction of DDG to FDCA

The present invention is directed to synthesizing 2,5-disubstituted furans (which may include, e.g., FDCA) by the dehydration of oxidized sugar products (which may include, e.g., DDG). In accordance with some aspects of the invention, the dehydration methods produce higher yields and/or higher parity 2,5-disubstituted furans than previously known dehydration reactions.

In certain aspects, the DDG may be a DDG salt and/or a DDG ester. For example, esters of DDG may include dibutyl ester (DDG-DBE). Salts of DDG may include DDG-2K, which is a DDG dipotassium salt. The FDCA may be an FDCA ester (e.g., FDCA-DBE), For example, a starting material of DDG-DBE may be dehydrated to produce FDCA-DBE. For ease of discussion, “DDG” and “FDCA” as used herein refer to DDG and FDCA generically (including but not limited to esters thereof), and not to any specific chemical form of DDG and FDCA. Specific chemical forms, such as esters of FDCA and DDG, are identified specifically.

DDG is dehydrated to produce FDCA. The dehydration reaction may additionally produce various byproducts in addition to the FDCA. In some aspects, DDG is combined with a solvent (e.g., an acidic solvent) and/or a catalyst, and allowed to react to produce FDCA. DDG may be dissolved in a first solvent prior to adding the DDG (i.e., the dissolved DDG and the first solvent) to a catalyst. In some aspects, DDG may be dissolved in a first solvent prior to adding the DDG to a catalyst and/or a second solvent. It is generally understood that by dissolving the DDG in a first solvent prior to adding any other component (e.g., a catalyst or reactant) causes a more efficient reaction, from FDCA to DDG. A few reasons for why a more efficient reaction may occur include, by dissolving DDG-2K in a solvent prior to adding a catalyst or acidic solvent the DDG-2K is more effective in solution; DDG may adopt its preferred form when first dissolved in a solvent; and DDG in solution may increase yields of FDCA.

In certain aspects, the catalyst is also a solvent. In some aspects, the catalyst also acts as a dehydrating agent. The catalyst may be a salt, gas, elemental ion, and/or an acid. In certain aspects, the catalyst and/or solvent is selected from one or more of elemental halogen (e.g., elemental bromine, elemental chlorine, elemental fluorine, elemental iodine, and the like), hydrohalic acid (e.g., hydrobromic acid, hydrochloric acid, hydrofluoroic acid, hydroiodic acid, and the like), alkali and alkaline earth metal salts (e.g., sodium bromide, potassium bromide, lithium bromide, rubidium bromide, cesium bromide, magnesium bromide, calcium bromide, strontium, bromide, barium bromide, sodium chloride, potassium chloride, lithium chloride, rubidium chloride, cesium chloride, magnesium chloride, calcium chloride, strontium chloride, barium chloride, sodium fluoride, potassium fluoride, lithium fluoride, rubidium fluoride, cesium fluoride, magnesium fluoride, calcium fluoride, strontium fluoride, barium fluoride, sodium iodide, potassium iodide, lithium iodide, rubidium iodide, cesium iodide, magnesium iodide, calcium iodide, strontium iodide, barium iodide, other alkali or alkaline earth metal salts, other salts in which at least some of the negative ions are halides, and the like), acetyl chloride, other acid alides or activated species, other heterogeneous acid catalysts, trifluoroacetic acid, acetic acid, n-methylpyrrolidone acid, propionic acid, butyric acid, formic acid, other ionic liquids, nitric acid, sulfuric acid, phosphoric acid, methanesulfonic acid, p-toluenesulfonic acid, other supported sulfonic acids (e.g., nation, Amberlyst®-15, other sulfonic acid resins, and the like), heteropoly acids (e.g., tungstosilicic acid, phosphomolybdic acid, phosphotungstic acid, and the like), acids with a first pKa<2, and other supported organic, inorganic, and supported or solid acids. A catalyst may be obtained from any source that produces that catalyst in a reaction mixture (e.g., a bromine containing catalyst may be obtained from any compound that produces bromide ions in the reaction mixture).

Acetic acid is a particularly desirable solvent as the ultimate FDCA product has a lower color value, e.g. it is whiter than products produced with other solvents. Trifluoroacetic acids is an additional preferred solvent for the production of FDCA.

It is generally understood that the dehydration of DDG to FDCA by the methods discussed herein provide molar yields of FDCA larger than those obtained from previously known dehydration reactions. In some aspects, the dehydration reaction yields at least 20%, at least 30%, at least 40%, at least 50%, at least 55%, at least 60%, at least 65%, at least 70%, at least 75%, at least 80%, at least 85%, at least 90%, at least 95%, or at least 99% molar yield of FDCA that may be produced from DDG as the starting material. In other aspects, the dehydration reaction yields between 20% and 100%, between 20% and 90%, between 20% and 80%, between 30% and 100%, between 30% and 90%, between 30% and 80%, between 40% and 100%, between 40% and 90%, between 40% and 80%, between 40% and 70%, between 40% and 60%, between 50% and 100%, between 50% and 90%, between 50% and 80%, between 50% and 70%, between 55% and 95%, between 55% and 90%, between 55% and 85%, between 55% and 80%, between 55% and 75%, between, 55% and 70%, between 60% and 99%, between 60% and 95%, between 60% and 90%, between 60% and 85%, between 60% and 80%, between 65% and 99%, between 65% and 95%, between 65% and 90%, between 65% and 85%, between 65% and 80%, between 70% and 99%, between 70% and 95%, between 70% and 90%, between 70% and 85%, between, 75% and 99%, between 75% and 95%, between 75% and 90%, between 75% and 85%, between 80% and 99%, between 80% and 95%, between 85% and 99%, or between 90% and 99% molar yield of FDCA that may be produced from DDG as the starting material.

The FDCA produced via the dehydration reaction may be isolated and/or purified. Suitable isolation or purification techniques include filtrating and washing the FDCA product with water or recrystallizing the FDCA from water.

The purified FDCA may have multiple uses in the industry such as an alternative to terephthalic acid in producing polyethylene terephthalate (PET). PET is commonly used to manufacture polyester fabrics, bottles and other packaging. FDCA may also be a precursor for adipic acid, jet fuels, other diols, diamine, or dialdehyde based chemicals.

In one aspect, the process described above is conducted by adding DDG and a catalyst and/or a solvent into a reaction vessel provided with a stirring mechanism and then stirring the reuniting mixture. The reaction vessel may be a batch or a continuous reactor. A continuous reactor may be a plug flow reactor, continuous stirred tank reactor, and a continuous stirred tank reactor in series. In some aspects, the reaction vessel may be selected for a dehydration reaction based on its metallurgy (e.g., a zirconium reactor may be selected over a teflon reactor). A reaction vessel may be a zirconium reactor, a teflon reactor, a glass-lined reactor, or the like. The temperature and pressure within the reaction vessel may be adjusted as appropriate. The DDG may be dissolved in a solvent prior to adding the DDG to the reaction vessel. In certain aspects, DDG is mixed with the solvent at a temperature within the range of 5° C. to 40° C., and in more specific aspects at about 25° C., to ensure dissolution in the solvent before the catalyst is added and reaction is initiated. Additionally and/or alternatively, the catalyst may be mixed with the solvent at room temperature to ensure dissolution in the solvent before being added to the DDG.

In some aspects, the process includes removing water produced during the reaction. Reducing at least some of the water produced may reduce or eliminate side reactions and reactivate the catalysts. As a consequence higher product yields may be obtained. Any suitable means may be used to regulate the amount of water in the reaction vessel such as use of a water content regulator.

The manufacturing process of FDCA may be conducted in a batch, a semi-continuous, or a continuous mode. In certain aspects, the manufacture of FDCA operates in a batch mode with increasing temperatures at predefined times, increasing pressures at predefined times, and variations of the catalyst composition during the reaction. For example, variation of the catalyst composition during reaction can be accomplished by the addition of one or more catalysts at predefined times.

The temperature and pressure typically can be selected from a wide range. However, when the reaction is conducted in the presence of a solvent, the reaction temperature and pressure may not be independent. For example, the pressure of a reaction mixture may be determined by the solvent pressure at a certain temperature. In some aspects, the pressure of the reaction mixture is selected such that the solvent in mainly in the liquid phase.

The temperature of the reaction mixture may be within the range of −20° C. to 180° C., and in certain aspects may be within the range of 20° C. to 100° C., and in more specific aspects at a temperature of 60° C. A temperature above 180° C. may lead to decarboxylation to other degradation products and thus such higher temperatures may need to be avoided.

In some aspects, a dehydration, reaction may run for up to 48 hours. In alternative aspects, a dehydration reaction may run for less than 5 minutes (i.e., the dehydration reaction is at least 95% complete within 5 minutes). In certain preferred examples, a dehydration reaction may occur within the time range of 1 minute to 4 hours, (i.e., the dehydration reaction of the reaction mixture is at least 95% complete within 1 minute to 4 hours). In some aspects the reaction of the reaction mixture is at least 95% complete within no more than 1 minute, 5 minutes, 4 hours, 8 hours or 24 hours. The length of the reaction process may be dependent on the temperature of the reaction mixture, the concentration of DDG, the concentration of the catalyst, and the concentration of other reactants. For example, at low temperatures (e.g., at or near the freezing point of the selected solvent) the reaction may run for up to two days, but at high temperatures (e.g., above 100° C.) the reaction may run for less than five minutes to achieve at least 95% completion.

Upon completion of the reaction process, a reaction product may be formed including FDCA and various byproducts. The term “byproducts” as used herein includes all substances others than 2,5-furandicarboxylic acid and water. In some aspects, the number, amount, and type of byproducts obtained in the reaction products may be different than those produced using other dehydration processes. Undesirable byproducts, such as 2-furoic acid and lactones, may be produced in limited amounts. For example, byproducts may include,

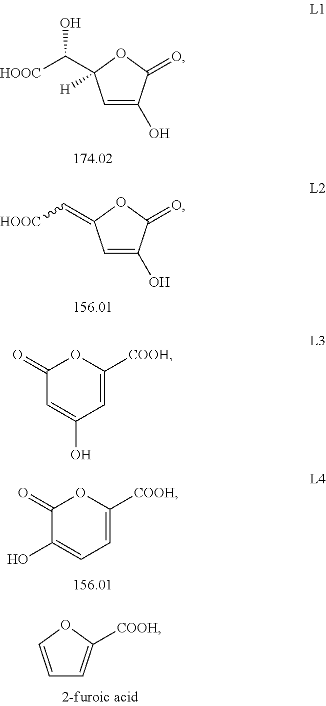

and the like. In certain aspects, undesirable byproducts may also include DDG-derived organic compounds containing at least one bromine atom. A reaction product may contain less than 15%, alternatively less than 12%, alternatively 10% to 12%, or preferably less than 10% byproducts. The reaction product may contain at least 0.5%, less than 7%, 0.5% to 5%, 5% to 7%, or about 5% lactone byproducts. “Lactone byproducts” or “lactones” as used herein include the one or more lactone byproducts (e.g., L1, L2, L3, and/or L4) present in the reaction product. Additionally or alternatively, the reaction product may contain less than 10%, 5% to 10%, or about 5% 2-furoic acid.

In certain aspects, the resulting FDCA may be isolated and/or purified from the reaction product. For example, the resulting FDCA may be purified by recrystallization techniques. In some aspects, the isolated and/or purified FDCA still includes small amounts of byproducts. The purified product may contain at least 0.1% (1000 ppm) lactone byproducts. In some aspects, the purified product contains less than 0.5% (5000 ppm), or preferably less than 0.25% (2500) lactone byproducts. In some aspects, the purified product contains between about 0.1% and about 0.5% lactone byproducts.

### Synthesis of FDCA Using as Anhydride

In an aspect of the invention, FDCA is synthesized from DDG in combination with a reactant. For example, DDG-DBE may be dehydrated to form FDCA-DBE;

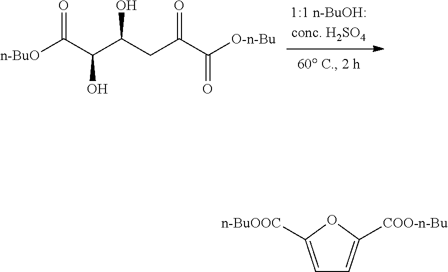

DDG may be combined with a reactant to form a reaction mixture. The reactant may be selected from an activated carboxylic acid derivative, activated sulfonic acid derivative, carboxylic acid halide, a ketene, or a combination thereof. In some aspects, the activated carboxylic acid derivatives act as both a catalyst and a solvent. An activated carboxylic acid derivative may include acetic anhydride, trifluoroacetic anhydride, acetyl chloride, acetyl bromide, and the like. In some aspects, an anhydride reactant acts as both a solvent and a catalyst (e.g., acetic anhydride). An activated sulfonic acid derivative may include methanesulfonyl chloride, tosyl chloride, triflic anhydride, chlorosulfonic acid, thionyl chloride, phosphoryl chloride, phosgene, and the like.

In certain aspects, a solvent may be added to the reaction mixture. The solvent may be selected from acetic acid, sulfuric acid, propionic acid, butyric acid, trifluoroacetic acid, formic acid, methanesulfonic acid, N-methylpyrrolidone, ionic liquids, or combinations thereof. Additionally or alternatively, a catalyst may be added to the reaction mixture. The catalyst may be selected from a halide salt (e.g., alkali metal halides, alkaline earth metal halides, transition metal halides, rare earth metal halides, or organic cations (e.g., quaternary ammonium ions, tertiary ammonium ions, secondary ammonium ions, primary ammonium ions, or phosphonium ions) in combination with halide ions), a hydrohalic acid, an elemental ion, an acid, and any combination thereof. The catalyst may be selected from sulfuric acid, phosphoric acid, methanesulfonic acid, sulfonic acid resin, hydrobromic acid, hydrochloric acid, hydrofluoroic acid, hydroiodic acid, other supported acids, hydrogen bromide, sodium, bromide, potassium bromide, lithium bromide, rubidium bromide, cesium bromide, magnesium bromide, calcium bromide, strontium bromide, barium bromide, FeBr₃, AlBr₃, NH₄Br, [EMIM]Br, sodium chloride, potassium chloride, lithium chloride, rubidium chloride, cesium chloride, magnesium chloride, calcium chloride, strontium chloride, barium chloride, FeCl₃, AlCl₃, NH₄Cl, [EMIM]Cl, sodium fluoride, potassium fluoride, lithium fluoride, rubidium fluoride, cesium fluoride, magnesium fluoride, calcium fluoride, strontium fluoride, barium fluoride, FeF₃, AlF₃, NH₄F, [EMIM]F, sodium iodide, potassium iodide, lithium iodide, rubidium iodide, cesium iodide, magnesium iodide, calcium iodide, strontium iodide, barium iodide, FeI₃, AlI₃, NH₄I, [EMIM]I, or any combination thereof. In some aspects, a catalyst and a solvent may be the same compound. For example, sulfuric acid, trifluoroacetic acid, and methanesulfonic acid may act as both a solvent and a catalyst.

Acetic anhydride may be used as a solvent and a catalyst. In other aspects, acetic anhydride as a reactant is used with a co-solvent (e.g., acetic acid). In some aspects, co-catalysts, such as acids and salts, are used to accelerate the reaction. In certain aspects, an acid catalyst used in combination with acetic anhydride triggers a faster and higher yielding reaction. Although not wishing to be bound by any particular theory, it is possible that the anhydride may react with the alcohol groups of the DDG to form acetyl esters, which are better leaving groups for the dehydration of the DDG to FDCA than the original hydroxyl groups.

Additional carboxylic acid anhydrides, which may include a single acid or mixed acids, may be used in a similar manner as acetic anhydrides (e.g., may act as solvent and catalyst, or may be used with a co-solvent and/or co-catalyst). Different anhydrides have different reactivity characteristics, which may correlate with the pKa of the corresponding acids and the steric bulk of the acid. For example, trifluoroacetic acid is very reactive and may fee used alone as a rapid dehydrating agent.

Carboxylic acid halides may be used in a similar manner as acetic anhydrides in the dehydration reaction of DDG to FDCA (e.g., may act as both solvent and catalyst, or may be used with a co-solvent and/or a co-catalyst). The reaction of the carboxylic acid halide with DDG forms hydrohalic acids (e.g., hydrobromic acid, hydrochloric acid, and the like). The reagents may produce a combined effect of acid catalyst and reagent. In certain aspects, the reactivity for the halides correlates to the pKa of the corresponding acids, the steric bulk of the acid, and the identity of the halide.

Activated sulfonic acid derivatives may be used in a similar manner as acetic anhydrides (e.g., may act as both solvent and catalyst, or may be used with a co-solvent and/or a co-catalyst). Activated sulfonic acid derivatives may include halides and/or anhydrides, and may include methanesulfonyl chloride, tosyl chloride, triflic anhydride, chlorosulfonic acid, thionyl chloride, phosphoryl chloride, phosgene, and the like. Additionally, ketene (e.g., ethenene) may also be used in a similar manner as the acetic anhydrides in the dehydration reaction of DDG to FDCA.

The reagents (e.g., DDG, catalyst, solvent) may be combined together in any suitable reaction vessel such as a batch or a continuous reactor. A continuous reactor may be a plug flow reactor, continuous stirred tank reactor, and a continuous stirred tank reactor in series. A reactor may be selected based on its metallurgy. For example, a reactor may be a zirconium reactor, a teflon reactor, a glass-lined reactor, or the like. A preferred reactor may be selected based upon corrosion and chemical compatibility with the reactant being utilized in the dehydration reaction. In some aspects, the reaction vessel is preheated prior to initiating a dehydration reaction.

In some aspects, DDG is dissolved in a solvent and then combined with a reactant to form a reaction mixture. The reaction of the reaction mixture may proceed at a temperature within a range of −20° C. to 200° C., alternatively within a range of 0° C. to 200° C., alternatively within a range of 20° C. to 100° C., or preferably within a range of 60° C. to 100° C. The pressure in the reaction vessel may be auto generated by the reaction components at the reaction temperature. In some aspects, the reaction may proceed (i.e., reach 95% completion) for up to two clays if the reaction temperature is low, or the reaction may proceed for less than five minutes if the temperature is 100° C. or higher. A preferred reaction time for the reaction mixture is within the range of one minute to four hours. The reaction may proceed to yield a reaction product including FDCA, water, and other byproducts (e.g., lactones). The FDCA may be filtered and removed from the reaction product.

In some aspects, the reaction may proceed at a fixed temperature, in alternative aspects, the temperature of the reaction mixture may be increased rapidly after the reaction mixture is formed. For example, the temperature of the reaction mixture may be increased from an ambient temperature or from no more than 30° C. to 60° C. or to at least 60° C. within two minutes, alternatively within 5 minutes, or within 20 minutes. In another example, the temperature of the reaction mixture may be increased from an ambient temperature or from no more than 30° C. to 100° C. or to at least 100° C. within two minutes, alternatively within 5 minutes, or within 20 minutes. A fast heat up time, as compared to a slow or gradual temperature increase, can limit and/or prevent side reactions from occurring during the reaction process. By reducing the number of side reactions that occur during the reaction process, the number of byproducts produced during the reaction is reduced. In certain aspects, any byproducts produced by the dehydration reaction are present at below 15%, alternatively less than 12%, alternatively 10% to 12%, or preferably less than 10%.

In some aspects, an anhydride reagent is added to the reaction mixture at a molar ratio of at least 1:1 with the DDG. In certain, aspects, increased molar yield of FDCA is obtained when anhydride reagent is added to the reaction mixture at a molar ratio within the range of 2:1 to 10:1 with DDG. An increased yield of FDCA may be obtained when anhydride reagent is added to the reaction mixture at a molar ratio not exceeding 10:1 with DDG. In some aspects, the amount of acid catalyst is varied. The amount of acid catalyst may be within the range of 0.1 M to 1 M concentration. For example, sulfuric acid may be added to the reaction mixture at a concentration of 0.6 M.

An anhydride reagent may be combined with an acid in a 1:1 molar ratio (e.g., acetic anhydride in combination with acetic acid at a 1:1 molar ratio). The anhydride may be combined with acetic acid at a ratio within a range of 1:10 to 3:1. In certain aspects, the anhydride combined with acetic acid does not exceed a molar ratio of 3:1.

In some preferred aspects, the reactant is trifluoroacetic anhydride. A reaction mixture may contain trifluoroacetic anhydride and a catalyst of sulfuric acid. For example, a reaction mixture may include 0.1 M to 1.0 M sulfuric acid. The reaction mixture including sulfuric acid and trifluorcacetic anhydride may produce a reaction product including FDCA, byproducts, and water. The reaction product may include up to 15% byproducts, and 60% to 99% molar yield FDCA. In some additional examples, a solvent of trifluoroacetic acid may be added to the reaction mixture. When trifluoroacetic acid is added to the reaction mixture, the trifluoroacetic anhydride may be combined with the trifluoroacetic acid in a 1:1 molar ratio, or in other examples, may be combined at a ratio within the range of 1:10 to 3:1.

Exemplary solvent/catalyst combinations include, but are not limited to, 1) acetyl chloride (AcCl) and sulfuric acid; 2) trifluoroacetic anhydride (TFAA) and sulfuric acid; 3) trifluoroacetic anhydride, trifluoroacetic acid, and sulfuric acid; 4) acetic anhydride (Ac2O) and sulfuric acid; 5) acetic anhydride, acetic acid, and sulfuric acid. Examples of exemplary process parameters, including a DDG starting material, a solvent, a catalyst, molarity of an acid, molarity of the DDG, reaction time, reaction temperature, molar yield of the FDCA, and any additional comments, such as the volume percent of any water added to the reaction mixture, can be seen in Table 1.

TABLE 1

| | | | [Acid], | [DDG], | | | FDCA | |
|---|---|---|---|---|---|---|---|---|
| Feed | Solvent | Catalyst | M | M | Time, h | Temp, C. | Yield | Comments |
| DDG | AcCl | H₂SO₄ | 0.86 | | 4 | 60 | 51.57 | |
| 2K | | | | | | | | |
| DDG | AcCl | H₂SO₄ | 0.86 | | 48 | ambient | 37.80 | |
| 2K | | | | | | | | |
| DDG | TFAA | H₂SO₄ | 0.9 | | 4 | 60 | 99.50 | TFAA |
| DBE | | | | | | | | solvent |
| DDG | TFAA | H₂SO₄ | 0.9 | | 48 | ambient | 68.22 | TFAA |
| DBE | | | | | | | | solvent |
| DDG | TFAA | H₂SO₄ | 0.9 | | 4 | 60 | 93.11 | TFAA in |
| DBE | | | | | | | | TFA solvent |
| DDG | TFAA | H₂SO₄ | 0.9 | | 4 | 60 | 92.69 | TFAA in |
| DBE | | | | | | | | TFA solvent |
| DDG | Ac₂O | H₂SO₄ | 0.86 | | 4 | 60 | 63.46 | Ac₂O |
| 2K | | | | | | | | solvent |
| DDG | Ac₂O | H₂SO₄ | 0.86 | | 48 | ambient | 65.24 | Ac₂O |
| 2K | | | | | | | | solvent |
| DDG | Acetic | H₂SO₄ | 0.86 | | 6 | 60 | 82.30 | Ac₂O in |
| 2K | | | | | | | | HAc solvent |
| DDG | Acetic | H₂SO₄ | 0.58 | 0.05 | 1 | 60 | 83.19 | AC₂O in |
| 2K | | | | | | | | HAc solvent |

Conditions for various alternative dehydration reactions utilizing DDG-2K as the starting material in combination with trifluoroacetic anhydride or acetic anhydride are provided in Table 2.

TABLE 2

| | | | | | | Molar |
|---|---|---|---|---|---|---|
| | | | | | | Yield of |
| | Acid | | Water | Temp | Time | FDCA |
| Solvent | (M) | | (vol %) | (° C.) | (h) | (%) |
| TFA:TFAA 1:1 | H₂SO₄ | (0.9) | 0 | 60 | 4 | 57 |
| Ac2O:HAc 1:1 | HBr | (2.9) | 0 | 60 | 6 | 45 |
| Ac2O:HAc 1:1 | H₂SO₄ | (0.8) | 0 | 60 | 6 | 82 |
| Ac2O:HAc 1:1 | H₂SO₄ | (0.8) | 0 | 20 | 48 | 65 |

### EXAMPLES

It will be appreciated that many changes may be made to the following examples, while still obtaining similar results. Accordingly, the following examples, illustrating embodiments of processing DDG to obtain FDCA utilizing various reaction conditions and reagents, are intended to illustrate and not to limit the invention.

### Example 1

DDG-DBE is combined with 2.9 M HBr in acetic acid/acetic anhydride (1:1). The reaction proceeds at 60° C. for 4 hours yielding 72% FDCA-DBE molar yield.

### Example 2

DDG-DBE is combined with 0.8 M H₂SO₄ acetic acid/acetic anhydride (1:1). The reaction proceeds at 60° C. for 4 hours yielding 72% FDCA-DBE molar yield.

### Example 3

DDG-DBE is combined with 0.8 M H₂SO₄ in acetic acid/acetic anhydride (1:1). The reaction proceeds at 20° C. for 48 hours yielding 77% FDCA-DBE molar yield.

### Example 4

DDG 2K is combined with 2.9 M HBr in acetic acid/acetic anhydride (1:1). The reaction proceeds at 60° C. for 6 hours yielding 45% FDCA molar yield.

### Example 5

DDG 2K is combined with 0.8 M H₂SO₄ in acetic acid/acetic anhydride (1:1). The reaction proceeds at 60° C. for 6 hours yielding 82% FDCA molar yield.

### Example 6

DDG 2K is combined with 0.8 M H₂SO₄ in acetic acid/acetic anhydride (1:1). The reaction proceeds at 20° C. for 48 hours yielding 65% FDCA molar yield.

### Example 7

DDG 2K is combined with 0.8 M H₂SO₄ in acetyl chloride. The reaction proceeds at 60° C. for 4 hours yielding 52% FDCA molar yield.

### Example 8

DDG-DBE is combined with trifluoroacetic acid/trifluoroacetic anhydride (1:1). The reaction proceeds at 60° C. for 4 hours yielding 99% FDCA-DBE molar yield.

### Example 9

DDG-DBE is combined with 0.9 M H₂SO₄ in trifluoroacetic acid/trifluoroacetic anhydride (1:1). The reaction proceeds at 60° C. for 4 hours yielding >99% FDCA molar yield.

Aspects of the disclosure have been described in terms of illustrative embodiments thereof. Numerous other embodiments, modifications, and variations within the scope and spirit of the appended claims will occur to persons of ordinary skill in the art from a review of this disclosure. For example, the steps illustrated in the figures may be performed in other than the recited order unless otherwise described, and one or more steps illustrated may be options in accordance with aspects of the disclosure.

1-34. (canceled)

35. A method of producing 2,5-furandicarboxylic acid comprising:
mixing a solution including 4-deoxy-5-dehydroglucaric acid and a first solvent with a reactant selected from the group consisting of an activated carboxylic acid derivative, an activated sulfonic acid derivative, a carboxylic acid halide, a ketene, and combinations thereof in a reaction vessel to form a reaction mixture;
increasing temperature of the reaction vessel to a temperature within a range of 0° C. to 200° C.;
allowing the 4-deoxy-5-dehydroglucaric acid to react in the presence of the reactant to produce a reaction product of 2,5-furandicarboxylic acid, water, and byproducts;
removing the water produced during the reaction continuously or periodically; and
removing the 2,5-furandicarboxlic acid from the reaction product,
wherein the reactant is selected from the group consisting of trifluoroacetic anhydride, acetic anhydride, acetyl chloride, acetyl bromide, and combinations thereof
wherein the reactant is present in the reaction mixture in at least a 2:1 molar ratio with 4-deoxy-5-dehydroglucaric acid,
wherein the reactant is dissolved in a second solvent, and
wherein the byproducts produced include lactones.

36. The method of claim 35, wherein the produced 2,5-furandicarboxylic acid has a yield of greater than 50 mol %.

37. The method of claim 35, wherein the reactant is acetic anhydride.

38. The method of claim 35, wherein the second solvent is trifluoroacetic acid.

39. The method of claim 35, wherein the reactant is trifluoroacetic anhydride and the second solvent is trifluoroacetic acid, and the trifluoroacetic anhydride and the trifluoroacetic acid are present in the reaction mixture in a ratio of 1:10 to 3:1.

40. A composition of 2,5-furandicarboxylic acid including at least 85 wt % 2,5-furandicarboxylic acid and at least one byproduct selected from one or more of 2-furoic acid and lactones, prepared by a method comprising:
mixing 4-deoxy-5-dehydroglucaric acid with a reactant selected from the group consisting of an activated carboxylic acid derivative, an activated sulfonic acid derivative, a carboxylic acid halide, ketene, and combinations thereof to form a reaction mixture; and
allowing the 4-deoxy-5-dehydroglucaric acid to react in the presence of the reactant to produce 2,5-furandicarboxylic acid, water and byproducts.
